(19) United States
(12) Patent Application Publication
(10) Pub. No.: US 2012/0160229 A1
(43) Pub. Date: Jun. 28, 2012

(54) ATHLETIC WARMING DEVICE

(76) Inventor: Loc Tieu, Irvine, CA (US)

(21) Appl. No.: 13/045,258

(22) Filed: Mar. 10, 2011

Related U.S. Application Data
(60) Provisional application No. 61/427,706, filed on Dec. 28, 2010.

(51) Int. Cl. A61F 7/00; H05B 1/00; F24J 1/00

(52) U.S. Cl. 126/204

(57) ABSTRACT

A device and method for use by athletes and others that desire to keep at least a portion of their body warm during rest periods is provided. The invention provides a confinement area which may be designed around a particular body part to provide warmth to that particular body part during rest periods. It is contemplated that the confinement area is part of a warming device having a plurality of materials that make up the device and has end portions that are adapted to fit around and secure the device to the particular body part thereby allowing warmth to be trapped within the confinement of the device. The device may have at least an outside portion and an inside portion whereby the inside portion is adapted to surround a particular body part and secure that body part from environmental factors and provide a sufficiently warm environment. The device may also have a plurality of bands on the end portions to secure the device to the individual utilizing it.

### PRIORITY CLAIM

This application claims priority to the earlier filed U.S. Provisional Application No. 61/427,706 filed on Dec. 28, 2010, entitled “Athletic Arm Warming Device.”

### FIELD OF THE INVENTION

The field of the invention is for an athletic aid device. More specifically, an embodiment relates to methods, apparatuses, and device that may assist in warming a particular part of the body during an athletic event.

### BACKGROUND

In today's athletic environments, most athletes are told by coaches, trainers and physicians to warm up before practice or before the game. Athletes do a variety of activities to warm-up such as jogging, walking, stretching, performing drills, lifting weights, riding a bicycle and the like. Typically, the reason why athletes warm up prior to engaging in physical activities is to loosen up cold, tight muscles, and to lubricate joints prior to physical activity. Additionally, warming up prior to physical activity can also increase blood flow in the body and may prevent and/or limit injuries that might otherwise occur if the athlete does not warm up. However, if the warm up is done improperly it can also hurt an athlete's performance and may predispose them to injury. The first question is why should an athlete warm up at all? Athletes should warm up to increase the success of having an optimum performance. What type of warm up can enhance athletic performances? A properly performed warm up can and will enhance an athletic performance.

Studies have shown that warming up before intense exercise will reduce surges in arterial blood pressure, and avoid shocking the heart into action. Gradually allowing the heart to adjust to high intensity workouts, gives the lungs and blood time to bring extra oxygen to the heart that will be beneficial for the short and long term health of the athlete.

When muscles initially contract, energy is released. However, the body is not very efficient at using that heat. Part of the energy released from heat goes toward energy production, some goes to warming up the tissue, and the rest is lost through the skin. As the tissue warms up, your heart rate increases to circulate the blood faster in an attempt to remove the heat, which spreads the heat to other parts of the body. With the heat from energy being released, the working muscle and the extra heat gained from warming the circulating blood tends to make the muscle tissue become warmer.

The result is the muscle will have increased elastic properties versus a cold muscle or even a muscle at normal body temperature. Elastic properties allow the muscle to return back to normal resting length after being stretched. The greater the elasticity of the muscle, the less potential to strain or tear the muscle, this also allows muscle to contract stronger and faster. The increase in blood temperature may only be 1 to 2 degrees above normal body temperature. The term warmer muscle is used as oppose to warmed up muscle because the muscle is at about 98 degrees before warming up and the body will not allow itself to get too much warmer, roughly the body likes 99 to 100 degrees for optimum athletic performance.

However, the problem that exists is that once the physical activity commences, there are often large periods of time when the athlete is not taking part in the activity, whether that is because the athlete is a substitute for another player, or because of the nature of the sport being played there is substantial downtime between the athlete's performing time. For example, many football players play only defensive roles or offensive rolls. Therefore, when an offensive play is happening, the defensive players are not involved and are resting on the sidelines. While this rest may be beneficial for recovery, the warming up effect may be lost, especially when the rest periods become long. Therefore, joints, muscles and other warmed areas of the body begin to cool, contract and become tight again. This causes the athlete to again warm up prior to entering the physical game. Alternatively, many athletes will attempt to keep warm on the sidelines by riding a stationary bike or the like. While this activity may keep the athlete warmed up and limber, it has the negative effect of also requiring them to utilize energy to keep warm, negating the resting effects while the athlete is not involved in the physical play of the sport.

Therefore, a need exists for an improved apparatus and method for keeping the muscles and/or joints of an athlete warm during the time when the athlete is not involved in the sporting activity. Additionally, a need also exists for an improved apparatus and method that may allow the athlete to utilize a device to keep certain parts of their body warm during rest periods without the need for further physical activity. Moreover, a need exists for an improved apparatus and method for keeping muscles and/or joints warm without the need for cumbersome devices that are hard to remove and/or encompass the entirety of, or a large portion of the body.

### SUMMARY OF THE INVENTION

Various embodiments describes a device and method for use by athletes and others that desire to keep at least a portion of their body warm during rest periods. An embodiment provides a confinement area which may be designed around a particular body part to provide warmth to that particular body part during rest periods. It is contemplated that the confinement area is part of a warming device having a plurality of materials that make up the device and has end portions that are adapted to fit around and secure the device to the particular body part thereby allowing warmth to be trapped within the confinement of the device. The device may have at least an outside portion and an inside portion whereby the inside portion is adapted to surround a particular body part and secure that body part from environmental factors and provide a sufficiently warm environment. The device may also have a plurality of bands on the end portions to secure the device to the individual utilizing it.

Among the many different possibilities contemplated, the device and system may be utilized by athletes that desire to keep a particular part of their body warm.

Additionally, in an exemplary embodiment, the device and method may allow for an individual to keep a particular portion of their body warm while resting.

In yet another exemplary embodiment, it is contemplated that the device and system may allow for an individual and/or an athlete to keep a particular portion of their body warm while resting and/or while waiting to resume play in a sporting event.

In another exemplary embodiment, it is contemplated that the device and system may be provided to provide warmth to an individual whereby the device may cover at least a portion of the human body.

Another exemplary embodiment is that the device and system may be provided to provide warmth to an individual whereby the device may have at least an end portion whereby the end portion is adapted to be in direct contact with the outside portion of the body of the individual thereby enclosing the space covered by the device.

In another exemplary embodiment a device and system may be provided whereby the device provides warmth to an individual and further wherein the device may have a plurality of end portions having a cuff whereby the cuff is adapted to encompass and enclose the area covered by the device thereby shielding the portion enclosed by the device from outside elements such as cold, liquids and other types of environmental factors.

Still another exemplary embodiment provides a device and method for providing warmth to an individual and/or athlete whereby the device may utilize a plurality of materials in its construction.

Yet another exemplary embodiment provides a device and a method for providing warmth to an individual and/or athlete whereby the device may be constructed of a plurality of materials including at least one material making up an outer portion of the device and another second material making up the inner portion of the device.

Another exemplary embodiment is that a system and method provide the device a method for providing warmth to an individual and/or athlete whereby the device may be constructed of different materials including at least one material that is waterproof and windproof.

A further exemplary embodiment contemplates that the device and method may allow for an individual to warm a particular part of their body during periods of rest whereby the particular part of the body may be a knee, elbow, wrist, hands, arms, shoulders, legs, feet and the like.

Further, a contemplated embodiment of the device and method is to provide a warming device which may have a plurality of materials and a plurality of cuffed end portions whereby the cuffed end portions are adapted to fit snuggly against an area of the body of the individual and the area between the cuffed portions are shielded from environmental factors.

Additionally, in an exemplary embodiment, a device and method is provided such that a warming device is provided whereby the warming device has at least a confinement portion and/or area which surrounds and supports an area of the body whereby the confinement area keeps that particular area of the body warm during rest periods. The confinement area is contained between the two end portions having cuffs that shut in the air in the confinement portion such that the air typically warmed by the sweat of the individual will keep the confinement area warm until the device is removed and the individual enters the realm of play for the sporting event.

In yet another exemplary embodiment, it is contemplated that the device and system may include cloth material such as a terry cloth. The cloth material may be in the interior or on the exterior of the device. For example, terry cloth might be used on the inside and/or the outside of the device.

In another exemplary embodiment, it is contemplated that the device and system may include fabric such as Sherpa fabric material on the inside.

Another exemplary embodiment may include titanium on the wristband and top and bottom portions. For example, titanium may be used on the cuffs.

Additionally, in an exemplary embodiment, the device and method may provide warmth by including a heat generating element.

For example, in an exemplary embodiment, the device and method may include one or more electrical heating elements, one or more chemical heating elements, or both.

In another exemplary embodiment, the device and method may include one or more heat storage elements that may be pre-heated.

For example, in an exemplary embodiment, the device and method may include an enclosure that includes grain, rice, or some other substance that may be pre-heated using, e.g., a microwave or other heating or warming device.

In yet another exemplary embodiment, it is contemplated that the device may include one or more pockets for receiving one or more heating elements, one or more heat storage element, or both.

In another exemplary embodiment, a system comprises a device that includes one or more pockets for receiving one or more heating elements, one or more heat storage element, or both. The system further comprises one or more heating elements, one or more heat storage element, or both.

These and other aspects of the present invention will become apparent to those skilled in the art after a reading of the following description of the preferred embodiment in combination with the figures.

### BRIEF DESCRIPTION OF THE DRAWINGS

FIG. 1 illustrates an embodiment of a warming device in accordance with an embodiment of the systems and methods described herein.

FIG. 2 illustrates an embodiment of a warming device in accordance with an embodiment of the systems and methods described herein that provide warmth by including a heat radiating element according to the systems and methods described herein.

FIG. 3 illustrates an embodiment of a warming device that includes pockets that might be used to receive a heat radiating element according to the systems and methods described herein.

FIG. 4 illustrates a cross section of another embodiment of a warming device according to the systems and methods described herein.

FIG. 5 is a flow diagram that illustrates an example method in accordance with an embodiment of the systems and methods described herein.

### DETAILED DESCRIPTION OF THE PREFERRED EMBODIMENT

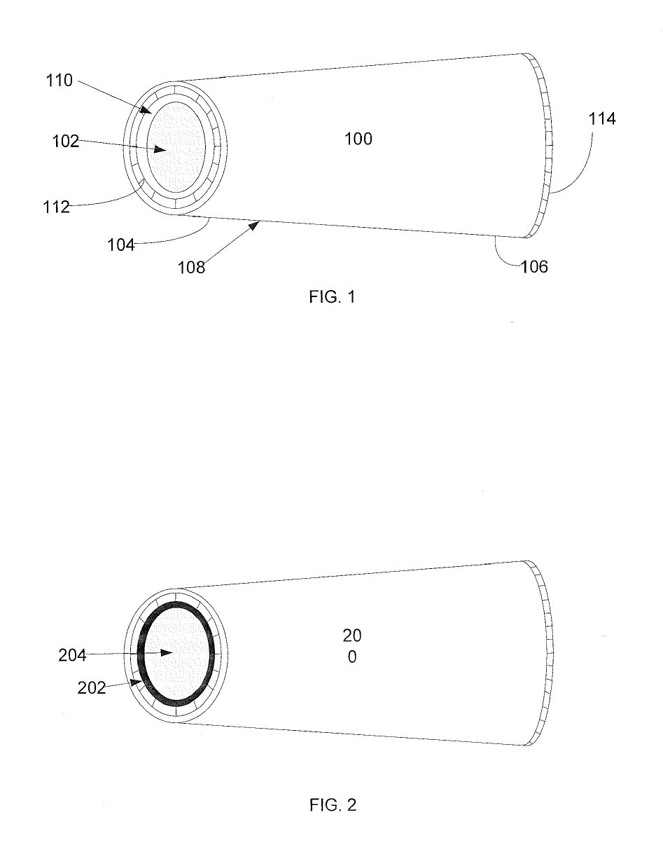

FIG. 1 is illustrates a top, front left side view of a warming device 100 in accordance with an embodiment of the systems and methods described herein. In the embodiment illustrated in FIG. 1 the warming device 100 includes a confinement area 102 which may be useful for warming or keeping warm various body parts, for example, an upper arm, forearm, lower leg, upper leg area, etc., depending on the size of the person using the device 100 and the size of the device 100 itself. While the example illustrated in FIG. 1 may generally be used for warming limbs or extremities, it will be understood that devices may be designed around any particular body part to provide warmth to that particular body part, e.g., during rest periods.

Warming device 100 can include a single material or a plurality of materials that make up the device. Some materials that might be used include spandex, terry cloth, Sherpa cloth, wool, etc. For example, certain devices 100 might include both a terry cloth outside portion 108 and a terry cloth inside portion 110. Other devices 100 might have a spandex outside portion 108 and a terry cloth inside portion 110, while still other devices 100 might have a terry cloth outside portion 108 and a wool or Sherpa fabric inside portion 110. Some embodiments may further include additional layers to provide for potentially greater retention.

In some embodiments, end portions 104, 106 that are adapted to fit around and secure the device 100 to the particular body part thereby allowing warmth to be trapped within the confinement of the device 100. The device 100 may have at least an outside portion 108 and an inside portion 110 whereby the inside portion 110 is adapted to surround a particular body part and secure that body part from environmental factors and provide a sufficiently warm environment.

The device 100 may also have a plurality of bands in the form of cuffs 112, 114 on the end portions to secure the device to the individual utilizing it. The illustrated embodiment of FIG. 1 includes one band 112, 114, on each end 104, 106 of the device 100. For example, the confinement area 102 is contained between the two end portions 104, 106 by cuffs 112, 114 that shut in the air in the confinement portion 102 such that the air typically warmed by body heat of the individual will keep the confinement area warm until the device 100 is removed and the individual enters the realm of play for the sporting event. Additionally, the plurality of cuffed end portions 112, 114 can be adapted to fit snuggly against an area of the body of the individual and the area between the cuffed portions 112, 114 to shield the area from environmental factors such as cold, wind, rain, snow, etc. While a single band is used on each end 106, 108, it will be understood that multiple bands may be used on each end 104, 106 to provide the confinement area 102 with a further seal against what may be colder air outside the confinement area 102.

The device 100 and system may be utilized by athletes that desire to keep a particular part of their body warm rather than the entire body generally or a large portion of the entire body. Accordingly, the device 100 can allow an athlete to keep a portion of the body warm while potentially generally avoiding overheating, for example. It will be noted that the device 100 of FIG. 1 is targeted to a specific portion of a person's body. For example the device 100 of FIG. 1 may allow for an individual to warm a knee, elbow, wrist, arms, legs, and the like. The device may allow for an individual to keep a particular portion of their body warm while resting or while waiting to resume play in a sporting event.

As illustrated in FIG. 1, end portion 104 has a smaller diameter than end portion 106 and the warming device 100 generally tapers from the larger diameter at end 106 to the smaller diameter at end 104. This shape allows the device 100 to generally follow the shape of the particular body part being warmed. For example, a person's arm area needs the wrist in generally smaller than the person's arm area further up the arm. Accordingly, having a tapered device 100 can be useful when the device is targeted to that portion of the body or other similarly shaped body parts.

FIG. 2 illustrates an example embodiment of a warming device 200 that provide warmth by including a heat radiating element 202 that might include a heat generating element, heat storage element, or both. Additionally, the heat generating element may be embedded between layers of the device 200 and might run from one end of the device 200.

In one example, the heat radiating element 202 might be a heat generating element such as an electrical heating pad, which might be battery powered, powered by AC electricity, or some other power source. In another example, the heat radiating element 202 might be a chemical heat source that includes substances that can be mixed together to produce an exothermic reaction that gives off heat to the surrounding device 200 and warms a confinement area 204. The heat from element 202 might generally be contained within the confinement area by the outer layer or layers of the device 200 and by the seal with the body part provides by the end cuffs.

In another example, the heat radiating element 202 may be some form of heat storage that slowly dissipates its heat to the confinement area 204. For example, the device 200 can include one or more heat storage elements that may be pre-heated. Materials that might be used as for heat storage include, but are not limited to chemical gels; grains, such as rice or wheat; water; or other substances. These substances do not generally produce heat, but rather, might store heat and dissipate it slowly over time. For example, in an exemplary embodiment, a grain such as rice or wheat may be enclosed within the device 200 and the entire device 200 might be placed into a microwave or other heating source to pre-heat the heat radiating element. Further, some substances may be used that can store cold or hot. Accordingly, if an injury occurs that may benefit from a “cold pack,” the device 200 might be used to hold such a cooling material near an injured area.

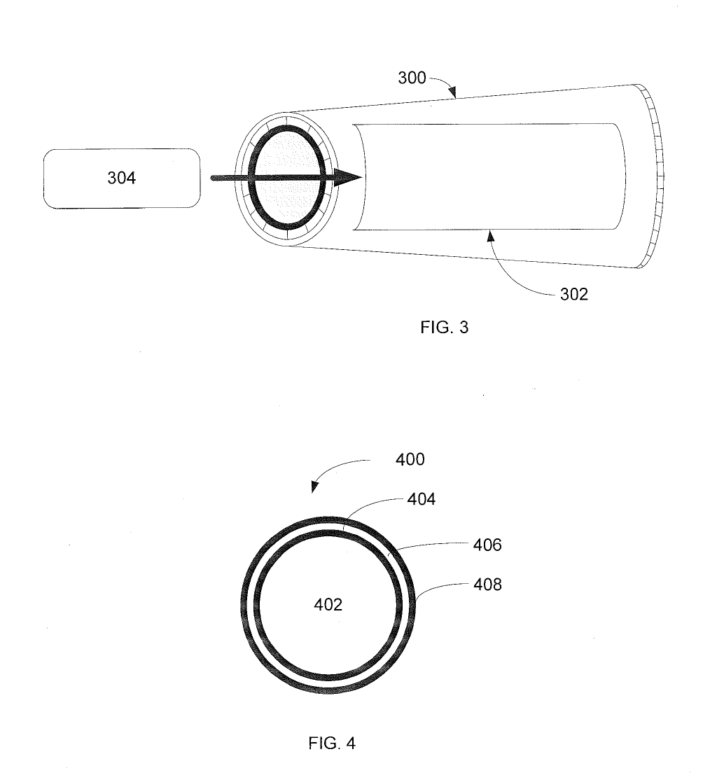

FIG. 3 illustrates an example embodiment of a warming device 300 that includes pockets 302 that might be used to receive a heat radiating element that might include a heat generating element, heat storage element, or both. As discussed above with respect to FIG. 2, in one example, the heat radiating element might be a heat generating element such as an electrical heating pad, which might be battery powered, powered by AC electricity, or some other power source. In another example, the heat radiating element might be a chemical heat source that includes substances that can be mixed together to produce an exothermic reaction that gives off heat to the surrounding device and warms a confinement area. The heat from the element might generally be contained within the confinement area by the outer layer or layers of the device and by the seal with the body part provides by the end cuffs.

In another example, the heat radiating element may be some form of heat storage that slowly dissipates its heat to the confinement area. For example, one or more heat storage element that may be pre-heated can be placed in pocket 302. As was discussed above, materials that might be used as for heat storage include, but are not limited to chemical gels; grains, such as rice or wheat; water; or other substances. These substances do not generally produce heat, but rather, might store heat and dissipate it slowly over time. For example, in an exemplary embodiment, a grain such as rice or wheat may be enclosed in a packet forming a separate heat storage element 304 that can be placed within the pocket 302 in the device 300. In some cases, the entire device 300 might be placed into a microwave or other heating source to pre-heat the heat storage element 304. Alternatively, the separate heat storage element 304, e.g., containing a chemical heat storage gel, grain, water, etc. might be heated independently of the device 300. Further, multiple elements 304 might be used so that as the elements 304 cool they can be replaced by warmer elements 304.

Substances may be used that can store cold or hot such that, if an injury occurs that may benefit from a “cold pack,” the device 300 might be used to hold such a cooling material near an injured area. Additionally, because pocket 302 is used in the embodiment illustrated in FIG. 3 “heat packs” and “cold packs” might be changed as needed with device 300. The “heat pack” may use materials particularly suited for heating properties, such as chemical packs that provide exothermic reactions or materials that store heat. The “cold pack” might be use materials that are particularly suited for cooling properties. These materials may provide endothermic chemical reactions to provided cooling or state changes such as ice or other frozen chemical melting.

FIG. 4 illustrates a cross section of another embodiment of a warming device 400 according to the systems and methods described herein. In the embodiment illustrated in FIG. 4 the warming device 400 includes a confinement area 402 which may be useful for warming or keeping warm various body parts, for example, an upper arm, forearm, lower leg, upper leg area, etc.

The device 400 may have at least an inside layer 404, an outside layer 408 and an area 406 for a heat radiating element. The area 406 can contain one or more heating elements, one or more heat storage element, or both. In some embodiments, the area 406 can be a pocket such that the heat radiating element is removable. The device 400 can provide warmth to an individual and/or athlete whereby the device may utilize a plurality of materials in its construction. For example, the different layers 404, 408 and area 406 may be made from different materials. The device may be constructed of a plurality of materials including at least one material making up an outer portion of the device and another second material making up the inner portion of the device.

In an embodiment, inside layer 404 can include cloth material such as a terry cloth, Sherpa fabric, spandex, etc. In another embodiment, outside later 408 can include cloth material such as a terry cloth, Sherpa fabric, spandex, etc. Further, some embodiments may include titanium on the wristband and top and bottom portions. For example, titanium may be used on cuffs of the device.

In another exemplary embodiment of the systems and methods described herein, the device may provide warmth to an individual and/or athlete whereby the device may be constructed of different materials including at least one material that is waterproof and windproof. These materials might be used for the outside layer 408.

Area 406 may provide warmth by including a heat generating element such as one or more electrical heating elements, one or more chemical heating elements, or both. In another exemplary embodiment, area 406 may include one or more heat storage elements that may be pre-heated. In another exemplary embodiment, area 406 can be one or more pockets for receiving one or more heating elements, one or more heat storage elements, or both.

Each of the devices 100, 200, 300, 400 illustrated may be used to provide warmth to an individual whereby the device may cover at least a portion of the human body. The devices 100, 200, 300, 400 can provide warmth to an individual whereby the device may have at least an end portion whereby the end portion is adapted to be in direct contact with the outside portion of the body of the individual thereby enclosing the space covered by the device 100, 200, 300, 400.

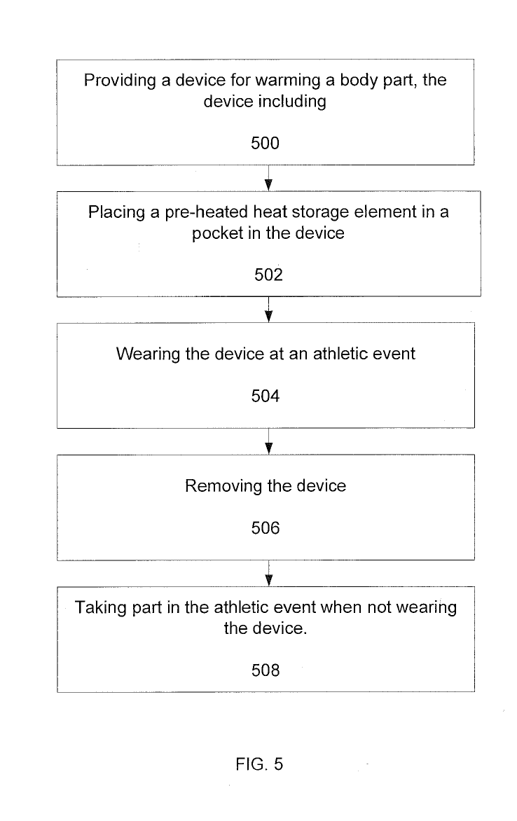

FIG. 5 is a flow diagram that illustrates an example method in accordance with an embodiment of the systems and methods described herein. In step 500 of the example method a device for warming a body part can be provided. The device may include a sleeve having a proximal and distal end, a confinement portion positioned between the proximal and distal end, and a plurality of cuffs defining the proximal and distal end whereby the plurality of cuffs having a diameter smaller than the confinement portion diameter. Additionally, the device can include a pocket that can be used to contain a heat storage element. If the device includes a pocket that may be used to contain a heat storage element the heat storage element can be pre-heated and placed in the pocket in step 502.

In step 504 a user wears the device at an athletic event. This can occur prior to the start of the athletic event, between plays, while the user is not an active player in the game, during a “time out,” at a break such as the end of a quarter, half, etc. In step 506 the user can remove the device prior to taking part in the athletic event while not wearing the device in step 508. In another embodiment that uses a device that includes a pocket, if the user is injured, the heating element may be removed and a cooling element, such as a “cold pack” can be used in its place.

By using the device during periods when an athlete is not actively playing a sporting event the user may maintain a warmed up state. The device can be used to target a specific muscle or groups of muscles. Additionally, because covering large portions of the body may be avoided, the user may be less likely to overheat.

In some embodiments, the device can be used in conjunction with a static stretching or a dynamic war-up routine. In a dynamic warm-up a person may do one or more of the following: (1) increase the core body temperature, (2) increase heart rate, (3) loosen joints, (4) spend time in an athletic position that might be used during the sporting activity, and (5) perform stretches. The stretches can include movements that develop balance and body awareness, include similar movements to the particular sport, and prepare for the skills that the person will perform during the sporting event. After such warm ups, the device described herein might be used to maintain preparedness for the sporting event so that an athlete does not cool down between activities of the sport.

While the invention is subject to various modifications and alternative forms, specific embodiments thereof have been shown by way of example in the drawings and will herein be described in detail. The invention should be understood to not be limited to the particular forms disclosed, but on the contrary, the intention is to cover all modifications, equivalents, and alternatives falling within the spirit and scope of the invention.

What is claimed is:

1. A device for warming a body part, the device comprising:
a sleeve having a proximal and distal end;
a confinement portion positioned between the proximal and distal end; and
a plurality of cuffs defining the proximal and distal end whereby the plurality of cuffs having a diameter smaller than the confinement portion diameter.

2. The device as recited in claim 1, wherein the plurality of cuffs have elastic materials incorporated therein to allow for contact of the plurality of cuffs with the outside edge of the body of the individual utilizing the device.

3. A device as recited in claim 1, wherein the device has at least an outside portion and an inside portion and wherein at least one of the outside portion and the inside portion includes spandex material, Sherpa material, or terry cloth.

4. A device as recited in claim 3, wherein the outside portion is constructed of one material and the inside portion is constructed of a second material.

5. A device as recited in claim 3, wherein the outside portion is constructed to withstand environmental forces and wherein the inside portion includes the confinement portion whereby the confinement portion is located closer to the individual user than the outside portion.

6. A device as recited in claim 1, wherein the confinement portion is in communication with a particular body part of an individual user.

7. A device as recited in claim 1 wherein the plurality of cuffs has elastic material to allow for fitment around a particular body part and may have a second beneficial material to improve blood flow and circulation to that particular body part.

8. A device as recited in claim 1 wherein the device is adapted for use around a particular body part including the elbow, aim, hand, wrist, shoulder, foot, knee, ankle, and the like.

9. A device as recited in claim 1 wherein device includes a terry cloth inside and a terry cloth outside.

10. A device as recited in claim 1 wherein device includes Sherpa fabric material within the device.

11. A device as recited in claim 1 further comprising titanium on the cuffs.

12. A device as recited in claim 1 further comprising a wrist band including titanium.

13. A device as recited in claim 1 further comprising a heat generating element.

14. A device as recited in claim 13 wherein the heat generating element comprises an electrical heating element.

15. A device as recited in claim 13 wherein the heat generating element comprises a chemical heating element.

16. A device as recited in claim 1 further comprising a heat storage element.

17. A device as recited in claim 16 wherein the heat storage element includes an enclosure that includes a heat storage material which is at least one of rice or wheat.

18. A device as recited in claim 1 further comprising a pocket for receiving one or more heating elements, heat storage element, or both.

19. A method comprising:
providing a device for warming a body part, the device including:
a sleeve having a proximal and distal end;
a confinement portion positioned between the proximal and distal end; and
a plurality of cuffs defining the proximal and distal end whereby the plurality of cuffs having a diameter smaller than the confinement portion diameter.
wearing the device at an athletic event;
removing the device; and
taking part in the athletic event when not wearing the device.

20. A method as recited in claim 19 wherein the device further comprises a pocket and wherein a heat storage element is pre-heated and placed in the pocket.
